(19) United States
(12) Patent Application Publication
(10) Pub. No.: US 2010/0016982 A1
(43) Pub. Date: Jan. 21, 2010

(54) TRAPEZIUM PROSTHESIS

(76) Inventor: Michael Wayne Solomons, Cape Town (ZA)

Correspondence Address: Arthur M Reginelli, 4 th Floor, First National Tower, Akron, OH, 44308, US

(21) Appl. No.: 12/518,191

(22) PCT Filed: Dec. 6, 2007
(86) PCT No.: PCT/ZA2007/000085

§ 371 Date: Jun. 8, 2009

(30) Foreign Application Priority Data
Dec. 7, 2006 (ZA) 2006/05686

(51) Int. Cl. A61F 2/42

(52) U.S. Cl. 623/21.11

(57) ABSTRACT

Reference numeral 10.1 generally depicts a prosthesis which includes an implant 12.1 which is configured to imitate or replicate the anatomic characteristics and functions of a trapezium bone 14 and a mounting means 16 for mounting the implant 12.1 on a FCR tendon 18 of a patient to inhibit dislocation of the implant 12.1 in use. The implant 12.1 is formed from any synthetic plastics or metallic material suitable for insertion in a CMC joint capsule 20. The implant 12.1 is contoured and/or dimensioned to facilitate engagement with and/or articulation of the trapezoid 22, scaphoid 24, index metacarpal 26, and thumb 28 metacarpal bones as shown in FIG. 1.

### FIELD OF THE INVENTION

This invention relates to a prosthesis, and more particularly, to a trapezium prosthesis.

### BACKGROUND TO THE INVENTION

Movement of a person's thumbs towards or into his palm, also known as opposition, is permitted by a joint known as the carpometacarpal (“CMC”) or basal joint. In particular, the CMC joint interconnects a metacarpal bone of the thumb and a trapezium bone of the wrist. The CMC joint is held in place by means of ligaments which also serve to form a joint capsule around the CMC joint. Articular cartilage on the CMC joint surface facilitates relative displacement or articulation between the thumb metacarpal and trapezium bones. When the articular cartilage wears out, the joint may develop a painful type of arthritis known as osteoarthritis.

The trapezium bone is also configured to articulate with the scaphoid, index metacarpal and trapezoid bones of the hand. A recess is defined in the trapezium bone for receiving a flexor Carpi radialis (FCR) tendon complementally therein, the recess further allowing the FCR tendon to be displaceable relative thereto.

One accepted surgical procedure to overcome this problem includes the excision and replacement of the trapezium bone with a rolled up slip of tendon. Another accepted surgical procedure includes the complete or partial replacement of the trapezium bone with a prosthetic implant such as the Wright Orthosphere. Although the above procedures have been successful, dislocation of the implant may still occur because of the inherent instability of the trapezeo metacarpal joint.

### SUMMARY OF THE INVENTION

According to the invention there is provided a prosthesis which includes:

an implant which is configured to imitate or replicate the anatomic characteristics and functions of a trapezium bone to be replaced; and

a mounting means for mounting the implant on a flexor carpi radialis (“FCR”) tendon of a patient thereby to inhibit dislocation of the implant in use.

The implant may be contoured and/or dimensioned to facilitate engagement with and/or articulation of the trapezoid, scaphoid, index metacarpal, and thumb metacarpal bones. The implant may be sized and dimensioned according to the trapezium bone to be replaced.

The mounting means may include a hook portion which is adapted to hook around a part of a FCR tendon and an extension portion which extends from the hook portion.

A bore may be defined in the implant for receiving at least a portion of the extension portion coaxially therein. A recess may be defined in the implant for receiving a free end of the hook portion to facilitate retention of the FCR tendon between the hook portion and the implant in an operative mounted or retaining condition of the prosthesis.

The extension portion may include a shaft portion which corresponds to the length of the bore, and a guide portion for guiding the shaft portion into the implant and towards the operative retaining condition. The guide portion may be detachably connectable to the shaft portion via any suitable connecting means. The connecting means may be in the form of an axially aligned screw threaded pin-and-socket arrangement.

A retaining means may be provided to retain the shaft portion in position in the bore of the implant in the operative retaining condition of the prosthesis.

The mounting means may include an elbow portion which is configured to hook around a part of the FCR tendon and arms which extend from the elbow portion. The arms may be dimensioned to be received in complementary registering bores defined in the implant.

The mounting means may have a longitudinally extending thermoplastic polymer core to enhance its rigidity. The thermoplastic polymer may be polyethylene terephthalate or Dacron.

A retaining means may be provided to retain the arms in position in the bores of the implant in the operative retaining condition of the prosthesis. The retaining means may be in the form of a generally annular shaped member which is configured to be received substantially co-axially the free end regions of the arms.

The annular shaped member may span the space between the arms and an inner surface of the bores and provide an interference fit therebetween in the operative mounted or retaining condition when the FCR tendon is held in position relative to the implant by the elbow.

According to a further aspect of the invention there is provided a prosthesis which includes:
a bearing member that is configured and dimensioned to replace a base of a thumb metacarpal and to permit complementary engagement and/or interdigitation with the implant; and
a connecting means for connecting the bearing member to the thumb metacarpal.

The connecting means may be in the form of a conventional morse taper.

The connecting means may be integral the bearing member.

The connecting means may be detachably connectable to the bearing member to permit replacement and/or adjustment of the bearing member.

The implant and/or mounting means and/or retaining means may be formed from any suitable synthetic plastics material which is inert within the human body. The plastics material may be any one or more of a selection of high density polyethylene (HDPE), low density polyethylene (LDPE), polyethylene terephthalate, polyvinyl chloride (PVC), polyurethane, nylon (polyamide) or Teflon (polytetrafluoroethylene or PTFE).

The implant and/or mounting means and/or retaining means may be formed from any suitable metallic material which is inert within the human body. The metallic material may comprise any one or more of a selection of copper, nickel, stainless steel, titanium, cobalt chrome or silicon.

### SPECIFIC EMBODIMENT

The invention will now be described by way of example with reference to the accompanying drawings.

In the drawings:

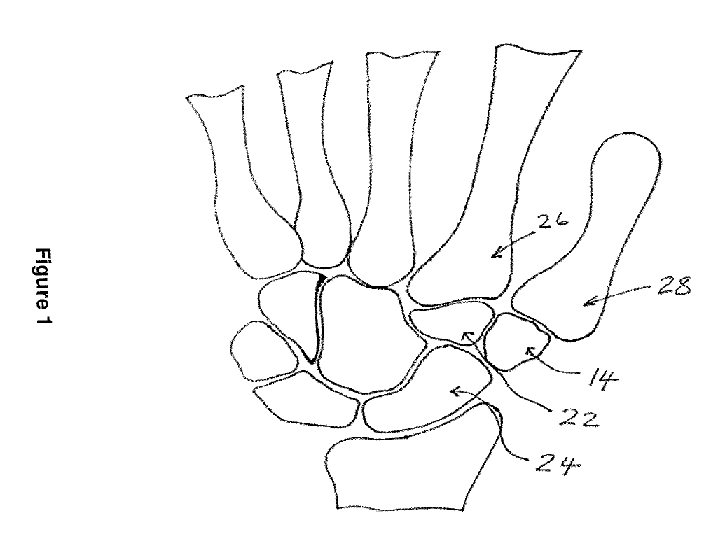

FIG. 1 is a schematic representation of bones forming a portion of a hand;

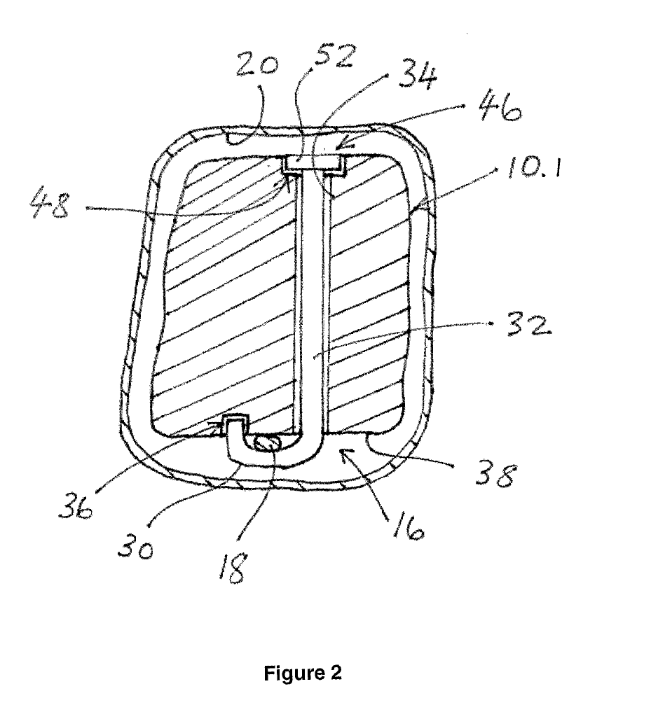

FIG. 2 is a sectioned schematic view of a prosthesis in accordance with the invention;

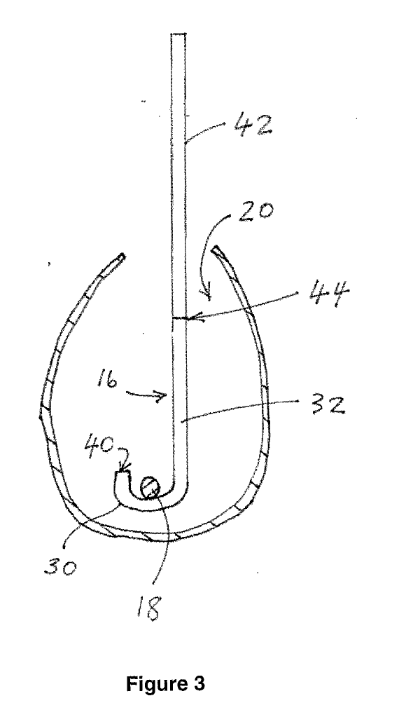

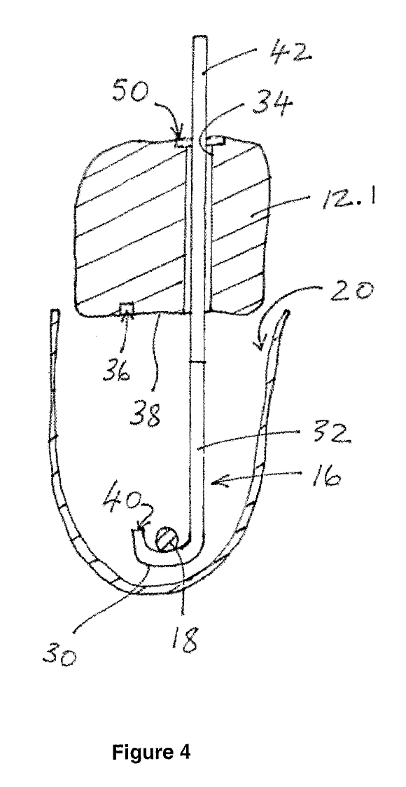

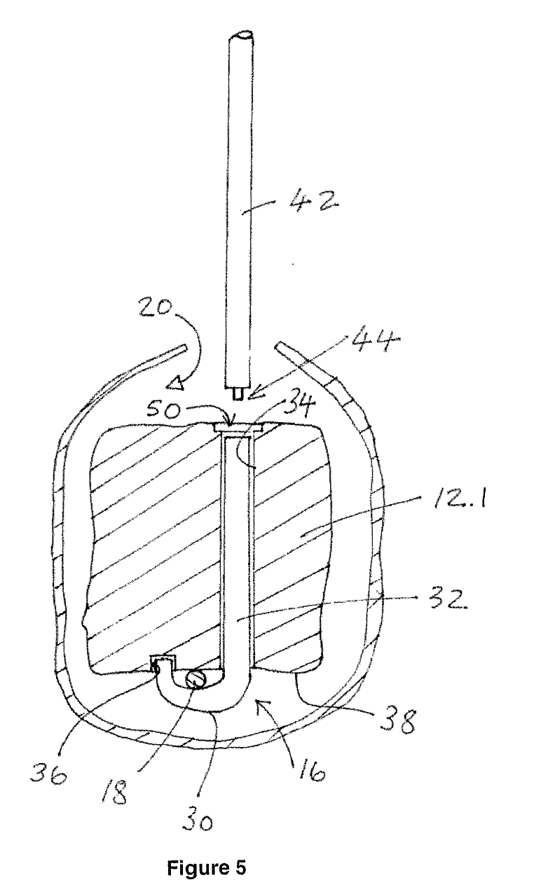

FIGS. 3 to 5 are sectioned schematic views of the progressive stages followed during implanting of the prosthesis;

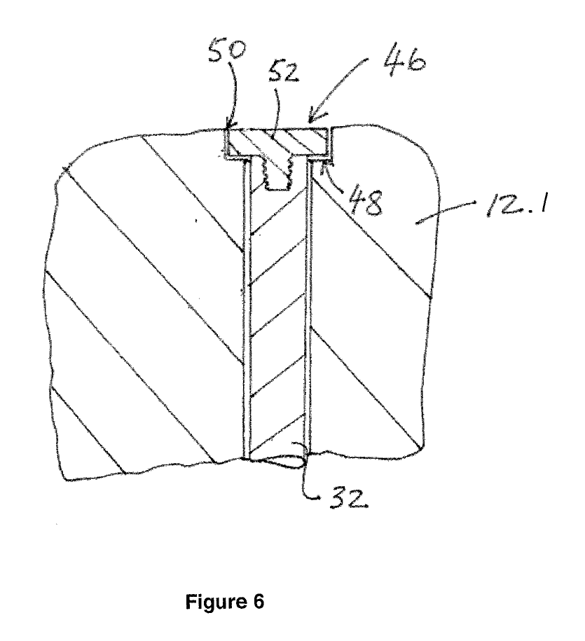

FIG. 6 is an enlarged sectioned view of a part of the prosthesis;

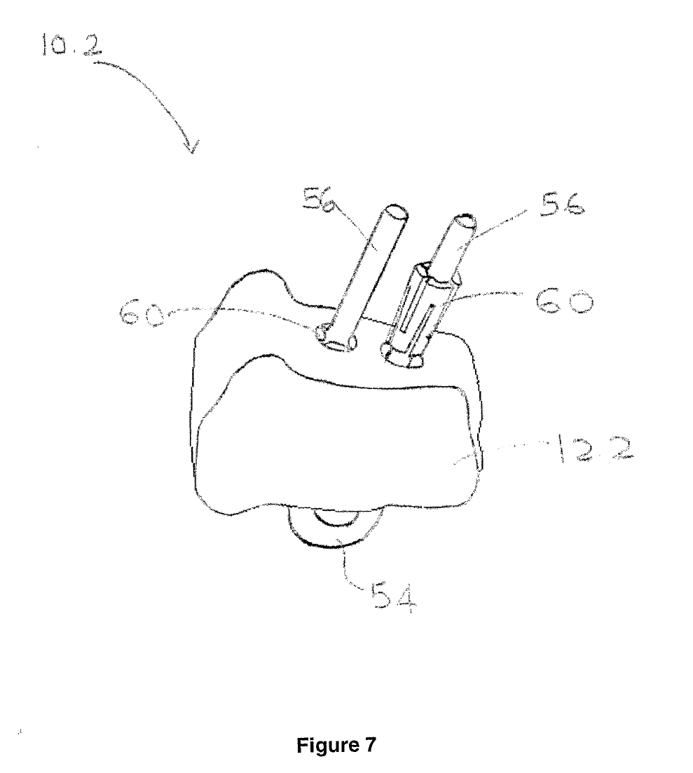

FIG. 7 is a three-dimensional schematic view of a further embodiment of a partially implanted prosthesis in accordance with the invention;

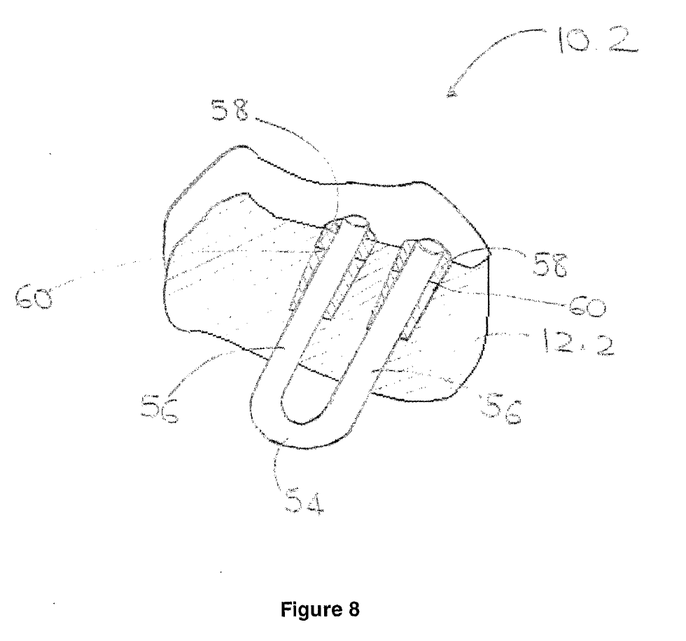

FIG. 8 is a sectioned view of the prosthesis shown in FIG. 7 in an operative mounted condition; and

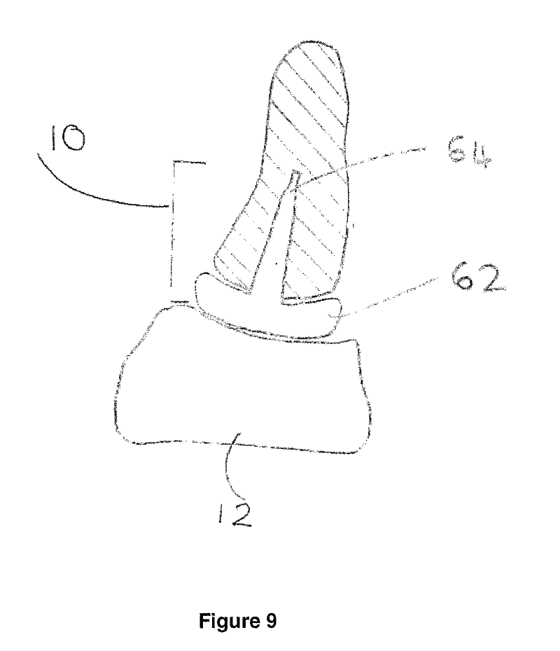

FIG. 9 is a plan view of a prosthesis incorporating a bearing member and connecting means in accordance with the invention.

Referring now to the drawings, reference numeral 10.1 generally depicts a prosthesis which includes an implant 12.1 which is configured to imitate or replicate the anatomic characteristics and functions of a trapezium bone 14 and a mounting means 16 for mounting the implant 12.1 on a FCR tendon 18 of a patient to inhibit dislocation of the implant 12.1 in use.

The implant 12.1 is formed from any synthetic plastics or metallic material suitable for insertion in a CMC joint capsule 20. The implant 12.1 is contoured and/or dimensioned to facilitate engagement with and/or articulation of the trapezoid 22, scaphoid 24, index metacarpal 26, and thumb 28 metacarpal bones as shown in FIG. 1. Typically, the implant 12.1 is sized and dimensioned according to the trapezium bone to be replaced.

The mounting means 16 includes a hook portion 30 which is adapted to hook around a part of the FCR tendon 18 and a shaft portion 32 which is dimensioned to be received in register a bore 34 defined in the implant 12.1. A recess 36 is defined in a base 38 of the implant 12.1 for receiving a free end 40 of the hook portion 30 to facilitate retention of the FCR tendon 18, and with it the implant 12.1, between the hook portion 30 and the implant 12.1 in an operative mounted or retained condition of the prosthesis 10.1.

A guide portion 42 for guiding the shaft portion 32 in to the bore 34 of the implant 12.1 and towards the operative retained condition is detachably connectable to the shaft portion 32 via an axially aligned screw threaded pin-and-socket arrangement 44.

A retaining means in the form of a fixing screw 46 is provided to retain the shaft receiving portion in position in the bore 34 of the implant 12.1 in the operative inserted condition. A recessed shoulder formation 48 is defined in the entrance 50 of the bore 34 to receive a head 52 of the screw 46 complementary therein.

According to another embodiment of the invention, as shown in FIGS. 7 and 8, the mounting means includes an elbow portion 54 which is configured to hook around a part of the FCR tendon, and arms 56 which extend from the elbow portion 54 and are dimensioned to be received in complementary registering bores 58 defined in an implant 12.2.

A retaining means in the form of an interference or friction fitting annular bush 60 is provided to retain the arms 56 in position in the bores 58 of the implant 12.2 in the operative retaining condition, as shown in FIG. 8. The annular bush 60 is configured to be received co-axially the free end regions of the arms and to span a space between the arms 56 and an inner surface of the bores 58. As shown in FIG. 8, the ends of the arms 56 are cut to size following implanting of prosthesis 12.2, so as not to protrude substantially beyond the surface of implant 12.2.

Referring now to FIG. 9, the prosthesis 10 further includes a bearing member 62 which is configured and dimensioned to replace a base of a thumb metacarpal 28 and to permit complementary engagement or interdigitation with the implant 12. The bearing member 62 is connected to a thumb metacarpal 28 via a connecting means in the form of a conventional morse taper 64.

The procedure of replacing the trapezium bone 14 involves a first step of opening the joint capsule 20 surrounding the CMC joint and excising the trapezium bone at the base of the thumb metacarpal 28. The surgeon then inserts the mounting means 16, which includes the guide portion 42, into the capsule 20 and hooks the hook portion 30 around the FCR tendon 18. Thereafter he slides the implant 12.1 down the guide portion 42 towards the shaft portion 32 until a position is reached wherein the tendon 18 is held captive between the hook portion 30 and the base 38. Once the guide portion 42 is screw threadedly detached from the shaft portion 32, the fixing screw 46 is screwed into a complementary screwed threaded socket formation defined in the free end of the shaft portion 32 to retain the tendon 18 in the captive condition.

Following excising of a part of a base of the thumb metacarpal 28, a longitudinally extending bore is drilled into the thumb metacarpal 28 for receiving complimentally, the morse taper 64. The bearing member 62 at the end of the morse taper 64 is then manipulated into mating engagement with the implant 12, to permit interdigitation or displacement therebetween. The capsule 20 and surrounding soft tissue are then sutured closed.

The applicant believes that the prosthesis 10 is advantageous in that because the implant 12 is attached to the FCR tendon 18, which is extremely strong and not prone to degenerate or rupture, the likelihood of dislocation of the implant 12 is greatly reduced.

The applicant further believes that the prosthesis 10 is advantageous in that the bearing member 62 serves to reduce pain caused by direct engagement between the thumb metacarpal 28 and the implant 12.

One must of course appreciate that the invention is not limited to the precise constructional details as hereinbefore described and which may be varied as desired.

1. A prosthesis which includes:
an implant which is configured, contoured and dimensioned to facilitate engagement and articulation with a trapezoid, scaphoid, index metacarpal and thumb metacarapal bones of a patient to allow the implant to imitate or replicate the anatomic characteristics and functions of a trapezium bone to be replaced; and
a mounting means for mounting the implant on a flexor carpi radialis (FCR) tendon of a patient thereby to inhibit dislocation of the implant in use.

2. (canceled)

3. A prosthesis as claimed in claim 1 in which the implant is sized and dimensioned according to the trapezium bone to be replaced.

4. A prosthesis as claimed in claim 2 in which the mounting means includes:
a hook portion that is adapted to hook around a part of the FCR tendon; and
an extension portion which extends from the hook portion.

5. A prosthesis as claimed in claim 4 in which a bore is defined in the implant for receiving at least a portion of the extension portion coaxially therein.

6. A prosthesis as claimed in claim 5 in which a recess is defined in the implant for receiving a free end of the hook portion to facilitate retention of the FCR tendon between the hook portion and the implant in an operative mounted or retaining condition of the prosthesis.

7. A prosthesis as claimed in claim 6 in which the extension portion includes:
a shaft portion which corresponds to the length of the bore; and
a guide portion for guiding the shaft portion into the implant and towards the operative retaining condition.

8. A prosthesis as claimed in claim 7 in which the guide portion is detachably connectable to the shaft portion via any suitable connecting means.

9. A prosthesis as claimed in claim 8 in which the connecting means is in the form of an axially aligned pin-and-socket arrangement.

10. A prosthesis as claimed in claim 9 in which the axially aligned pin and socket arrangement is screw threadedly connected.

11. A prosthesis as claimed in claim 10 in which a retaining means is provided to retain the shaft portion in position in the bore of the implant in the operative retaining condition of the prosthesis.

12. A prosthesis as claimed in claim 1 in which the mounting means includes:
an elbow portion which is configured to hook around a part of the FCR tendon and arms extending from the elbow portion, which arms are dimensioned to be received in complementary registering bores defined in the implant.

13. A prosthesis as claimed in claim 12 in which a retaining means is provided to retain the arms in position in the bores of the implant in the operative retaining condition of the prosthesis.

14. A prosthesis as claimed in claim 13 in which the retaining means is in the form of a generally annular shaped member which is configured to be received substantially co-axially the free end regions of the arms and to span the space between the arms and an inner surface of the bores to provide an interference fit therebetween in an operative mounted or retaining condition when the FCR tendon is held in position relative to the implant by the elbow.

15. A prosthesis as claimed in claim 1 which includes:
a bearing member that is configured and dimensioned to replace a base of a thumb metacarpal and to permit complementary engagement or interdigitation with the implant; and
a connecting means for connecting the bearing member to the thumb metacarpal.

16. A prosthesis as claimed in claim 15 in which the connecting means is a conventional morse taper.

17. A prosthesis as claimed in claim 16 in which the connecting means is integral the bearing member.

18. A prosthesis as claimed in claim 17 in which the connecting means is detachably connectable to the bearing member to permit replacement and/or adjustment of the bearing member.

19. A prosthesis as claimed in claim 18 wherein any one or more of the implant, mounting means and retaining means is formed from any suitable synthetic plastics material which is inert within the human body.

20. A prosthesis as claimed in claim 19 wherein the material is selected from any one or more of the group comprising high density polyethylene (HDPE), low density polyethylene (LDPE), polyethylene terephthalate, polyvinyl chloride (PVC), polyurethane, nylon (polyamide) or Teflon (polytetrafluoroethylene or PTFE).

21. A prosthesis as claims in claim 19 wherein the mounting means has a longitudinally extending thermoplastic polymer core to enhance its rigidity.

22. A prosthesis as claimed in claim 21 in which the core is polyethylene, terephthalate, or Dacron.

23. A prosthesis as claimed in claim 18 wherein any one or more of the implant, mounting means and retaining means is formed from any suitable metallic material which is inert within the human body.

24. A prosthesis as claimed in claim 23 wherein the material is selected from any one or more of the group comprising copper, nickel, stainless steel, titanium, cobalt chrome, or silicon.

25. (canceled)

26. (canceled)
